(19) United States
(12) Patent Application Publication
(10) Pub. No.: US 2024/0115496 A1
(43) Pub. Date: Apr. 11, 2024

(54) A COMPOUND DRY POWDER INHALANT AND USE THEREOF

(71) Applicant: TIANJIN UNIVERSITY OF TRADITIONAL CHINESE MEDICINE, Tianjin (CN)

(72) Inventors: Zhidong LIU, Tianjin (CN); Dongli QI, Tianjin (CN); Hui PENG, Tianjin (CN); Jiaxin PI, Tianjin (CN); Pan GUO, Tianjin (CN); Xiuping DENG, Tianjin (CN); Jiawei LI, Tianjin (CN)

(73) Assignee: TIANJIN UNIVERSITY OF TRADITIONAL CHINESE MEDICINE, Tianjin (CN)

(21) Appl. No.: 18/003,946

(22) PCT Filed: Jul. 27, 2022
(86) PCT No.: PCT/CN2022/108095

§ 371 Date: Dec. 30, 2022

(30) Foreign Application Priority Data
Aug. 25, 2021 (CN) 202110981774.7

(51) Int. Cl. A61K 9/00; A61K 31/137; A61K 31/198; A61K 31/7048; A61P 11/00

(52) U.S. Cl. CPC A61K 9/0075; A61K 31/137; A61K 31/198; A61K 31/7048; A61P 11/00

(57) ABSTRACT

The present application provides a compound dry powder inhalant, comprising baicalin, ambroxol hydrochloride, L-leucine and phosphate, wherein based on a mass of the compound dry powder inhalant, L-leucine accounts for 0-50%, phosphate accounts for 15-35%, and a total mass of baicalin and ambroxol hydrochloride accounts for 15-85%, and wherein a mass ratio of baicalin to ambroxol hydrochloride is 1:0.2 to 2. The compound dry powder inhalant has a Dv90≤5 μm. The drug combination of baicalin and ambroxol hydrochloride can effectively reduce inflammation and oxidative damage in lung tissue, alleviate pulmonary edema and histopathological changes, and reduce pulmonary dysfunction and pulmonary fibrosis.

The present application claims the priority of Chinese Patent Application No. 202110981774.7, filed before CNIPA on Aug. 25, 2021, titled “A COMPOUND DRY POWDER INHALANT AND USE THEREOF”, which is incorporated herein by reference in its entirety.

### FIELD OF THE INVENTION

The present application relates to the field of pharmaceutical technology, and in particularly relates to a compound dry powder inhalant and use thereof.

### BACKGROUND OF THE INVENTION

Idiopathic pulmonary fibrosis (IPF) is a lung disease of unknown etiology that tends to occur in middle-aged and elderly people, which is a chronic and irreversible development of diffuse alveolitis and structural disorders of the alveoli, characterized by extensive pulmonary remodeling due to the abnormal deposition of extracellular matrix, leading to pulmonary fibrosis (PF). The cause of most pulmonary fibrosis is unknown. Idiopathic pulmonary fibrosis is characterized by diffuse alveolitis and structural disorders of the alveoli, which eventually leads to pulmonary fibrosis. The development of IPF includes lung injury, inflammation, myofibroblast formation and accumulation of extracellular matrix, ultimately leading to structural dysfunction of lung. The pathogenesis is still unclear, and it is generally believed that inflammatory response and oxidative stress may be important factors involved in the initiation and progression of IPF. Clinical symptoms are insidious onset, without obvious early symptoms, initially presenting as coughing or coughing with phlegm, aggravated respiratory distress at late stage, leading to respiratory failure and death. IPF has a high mortality rate and is known as a tumor-like disease. Because IPF tends to occur in middle-aged and elderly people, the incidence of the disease is increasing globally as the aging of populations in various countries. According to the United World IPF Association, approximately 3.2 million people suffer from IPF, with 1.22 million new cases each year.

The treatment options proposed in the IPF clinical guidelines suggest that lung transplantation is the most direct and effective treatment for IPF. However, due to its high cost, fewer donors, and high technical risk, it is only acceptable to a few patients. Clinical treatment with glucocorticoids in combination with immunosuppressants and antioxidants is not recommended due to the unsatisfactory results and multiple toxic side effects. In the latest guidelines, the chemotherapeutic agents nintedanib and pirfenidone are listed as conditionally recommended for clinical treatment for IPF. However, there are some adverse reactions associated with long-term use, such as gastrointestinal discomfort, liver function impairment, and allergic skin reactions. Therefore, it is urgent to continue the search for efficacious and safe therapeutic drugs and treatments, while elucidating the pathophysiological mechanisms of pulmonary fibrosis.

In addition, the current clinical prevention and treatment of IPF is mainly oral administration and intravenous injection. Oral administration has gastrointestinal tract irritation and liver first pass effect, with low drug concentration in the blood, resulting in low bioavailability, and the efficacy is not apparent. Due to the long treatment cycle, the selection of injection has deficiencies such as poor patient compliance, low drug targeting, systemic toxicity and the like. Therefore, how to improve the targeting and bioavailability of drugs and achieve therapeutic effects in multiple aspects has become a long-standing common goal for pharmaceutical researchers.

### SUMMARY OF THE INVENTION

In the present application, it is found in a study that a drug combination of baicalin (BA, molecular formula as shown in Formula I) and ambroxol hydrochloride (AH, molecular formula as shown in Formula II), which is formulated into a dry powder inhalant (DPI) and administered via pulmonary inhalation can improve and treat pulmonary fibrosis by reducing the expression of inflammatory factors, improving lung injury and enhancing lung function. The present application is completed on this basis.

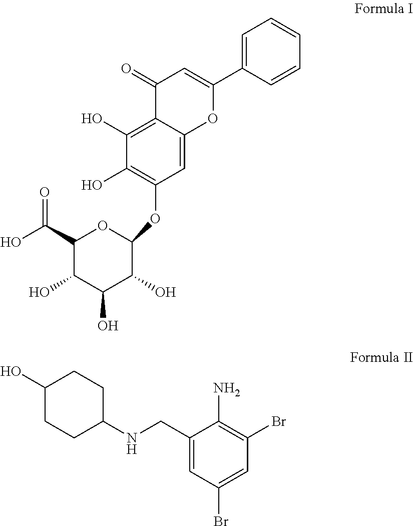

The first aspect of the present application provides a compound dry powder inhalant, comprising baicalin, ambroxol hydrochloride, L-leucine and phosphate, wherein based on a mass of the compound dry powder inhalant, L-leucine accounts for 0-50%, phosphate accounts for 15-35%, and a total mass of baicalin and ambroxol hydrochloride accounts for 15-85%, and wherein a mass ratio of baicalin to ambroxol hydrochloride is 1:0.2 to 2, preferably 1:0.8 to 1.2. The compound dry powder inhalant has a Dv90≤5 μm.

The second aspect of the present application provides use of the compound dry powder inhalant provided in the first aspect of the present application in the manufacture of a medicament for treating pulmonary interstitial fibrosis.

The present application provides a compound dry powder inhalant and its use in the manufacture of a medicament for treating pulmonary interstitial fibrosis, wherein a drug combination of baicalin and ambroxol hydrochloride can effectively reduce inflammation and oxidative damage in lung tissue, alleviate pulmonary edema and histopathological changes, and reduce pulmonary dysfunction and pulmonary fibrosis. Further, pulmonary administration of the compound dry powder inhalant will not only significantly increase the plasma half-life time and in vivo retention time of the drug, but also improve the bioavailability of the drug in the lung tissue, effectively reduce the clearance of the drug in the lung tissue, prolong the retention time of the drug in the lung, and facilitate the full effect of the drug in the lung tissue.

### BRIEF DESCRIPTION OF THE DRAWINGS

In order to more clearly illustrate the technical solution of the examples of the present application or the prior art, the drawings used in the examples and the prior art will be briefly described below. It is obvious to those skilled in the art that the drawings in the following description are only some examples of the present application, and other drawings may be also obtained according to these drawings.

FIG. 1 shows the moisture content of the BA/AH-DPI dry powder particles with different ratio.

FIG. 2 shows the moisture profile of the BA/AH-DPI dry powder particles with different ratio.

FIG. 3 shows the scanning electron micrograph of BA, AH and L-leu raw material.

FIG. 4 shows the scanning electron micrograph of the BA/AH-DPI dry powder particles with different ratio.

FIG. 5A shows the particle size distribution of BA particles.

FIG. 5B shows the particle size distribution of AH particles.

FIG. 5C shows the particle size distribution of L-Leu raw material.

FIG. 5D shows the particle size distribution of the BA/AH-DPI dry powder particles with 0% L-leu content.

FIG. 5E shows the particle size distribution of the BA/AH-DPI dry powder particles with 10% L-leu content.

FIG. 5F shows the particle size distribution of the BA/AH-DPI dry powder particles with 15% L-leu content.

FIG. 5G shows the particle size distribution of the BA/AH-DPI dry powder particles with 20% L-leu content.

FIG. 5H shows the particle size distribution of the BA/AH-DPI dry powder particles with 25% L-leu content.

FIG. 5I shows the particle size distribution of the BA/AH-DPI dry powder particles with 40% L-leu content.

FIG. 5J shows the particle size distribution of the BA/AH-DPI dry powder particles with 50% L-leu content.

FIG. 6 shows the DSC analysis profile of AH, BA, L-leu, a physical mixture thereof and the BA/AH-DPI dry powder of Example 4.

FIG. 7 shows the XRD schematic of AH, BA, L-leu and a physical mixture thereof.

FIG. 8 shows the XRD schematic of BA/AH-DPI dry powder particles with different ratio.

FIG. 9A shows the FPF comparison of BA in the BA/AH-DPI dry powder with different ratio.

FIG. 9B shows the FPF comparison of AH in the BA/AH-DPI dry powder with different ratio.

FIG. 10A shows the deposition rate of BA in the BA/AH-DPI dry powder with different ratio in each layer of NGI.

FIG. 10B shows the deposition rate of AH in the BA/AH-DPI dry powder with different ratio in each layer of NGI.

FIG. 11 shows the changes in body weight of rats in each group at days 1, 7, 14, 21 and 28.

FIG. 12 shows the lung factors of rats in each group after 28 days.

FIG. 13 shows the results of HE staining of rat lung tissue in each group.

FIG. 14 shows a graph of the total protein concentration in the alveolar lavage fluid of rats in each group.

FIG. 15 shows the expression levels of inflammatory factors IL-4, IL-6, IL-8, and IL-1β in BALF of rats in 8 groups.

FIG. 16 shows the expression of SOD, MDA, LDH and Hyp in rat lung tissue.

FIG. 17 shows the results of the comparison of MPO contents in the serum of rats in each group.

FIG. 18 shows the concentration profile of BA in plasma (ng/mL, Mean±SD, n=6).

FIG. 19 shows the concentration profile of AH in plasma (ng/mL, Mean±SD, n=6).

FIG. 20 shows the concentration profile of BA in lung tissue homogenate (ng/mL, Mean±SD, n=6).

FIG. 21 shows the concentration profile of AH in lung tissue homogenate (ng/mL, Mean±SD, n=6).

### DETAILED DESCRIPTION OF THE INVENTION

In order to make the objects, technical solutions, and advantages of the present application more clear and comprehensible, the present application will be further described in detail below with reference to the accompanying drawings and examples. It is apparent that the described examples are only a part of the examples of the present application, not all of examples. All other examples obtained by those ordinary skilled in the art based on the examples of the present application without making inventive efforts are within the scope of the present application.

The first aspect of the present application provides a compound dry powder inhalant (BA/AH-DPI), comprising baicalin, ambroxol hydrochloride, L-leucine and phosphate, wherein based on a mass of the compound dry powder inhalant, L-leucine accounts for 0-50%, phosphate accounts for 15-35%, and a total mass of baicalin and ambroxol hydrochloride accounts for 15-85%, and wherein a mass ratio of baicalin to ambroxol hydrochloride is 1:0.2 to 2, preferably 1:0.8 to 1.2. The compound dry powder inhalant has a Dv90≤5 μm.

The inventors find in a study that a drug combination of baicalin and ambroxol hydrochloride can effectively reduce inflammation and oxidative damage in lung tissue, alleviate pulmonary edema and histopathological changes, and reduce pulmonary dysfunction and pulmonary fibrosis. Further, the present application uses L-leucine (L-leu) as a DPI carrier, which is beneficial to improve the flowability and aerosol properties of the powder. Further, the inventors also find that L-leucine as the DPI carrier also has advantages including moisture resistance, improved dispersibility, and manufacturability.

In some embodiments of the first aspect of the present application, based on the mass of the compound dry powder inhalant, L-leucine accounts for 10-40%.

In some embodiments of the first aspect of the present application, based on the mass of the compound dry powder inhalant, L-leucine accounts for 15-25%.

The inventors find that when a content of L-leucine is within the above preferred range, it is beneficial to further improve the particle uniformity as well as the nebulization performance of the dry powder inhalant.

In some embodiments of the first aspect of the present application, based on the mass of the compound dry powder inhalant, a total content of BA and AH is 15-85%, preferably 40-60%.

In the present application, the compound dry powder inhalant is prepared with PBS (phosphate buffered saline solution) as a solvent. The obtained compound dry powder inhalant contains phosphate. Based on the mass of the compound dry powder inhalant, a content of the phosphate is 15-35%.

The PBS solution described in the application is a PBS buffer commonly used in the art and can be prepared by those skilled in the art according to existing formulations. The PBS solution usually comprises Na₂HPO₄, KH₂PO₄, NaCl and KCl. Therefore, the phosphate described in the present application can comprise a mixture of Na₂HPO₄, KH₂PO₄, NaCl and KCl.

To the extent that the effects of the present application are not impaired, unavoidable impurities may be additionally included.

In some embodiments of the first aspect of the present application, the compound dry powder inhalant has a Dv90≤3 μm.

In some embodiments of the first aspect of the present application, baicalin has a fine particle fraction of 30-60%; and ambroxol hydrochloride has a fine particle fraction of 25-50%.

In some embodiments of the first aspect of the present application, a mass median aerodynamic diameter of baicalin is 2-3.5 μm; and a mass median aerodynamic diameter of ambroxol hydrochloride is 2-3.5 μm.

In some embodiments of the first aspect of the present application, based on the mass of the compound dry powder inhalant, a moisture content is less than 7%.

In some embodiments of the first aspect of the present application, the compound dry powder inhalant is prepared by a spray drying.

In some embodiments of the first aspect of the present application, the spray drying comprises an inlet temperature of 75-85° C., an air flow of 80-120 L/min, a pump speed of 20-25%, a spray rate of 50-70%, and an internal pressure of 30-35 mbar.

The inventors find that the compound dry powder inhalant prepared by the method of the present application facilitates the reduction of powder cohesion, which improves the flowability and nebulization properties of the drug particles and ensures the consistency of aerosol properties between the drug and excipients.

The second aspect of the present application provides use of the compound dry powder inhalant provided in the first aspect of the present application in the manufacture of a medicament for treating idiopathic pulmonary fibrosis.

The inventors find in a study that in the compound dry powder inhalant of the present application, the drug combination of baicalin and ambroxol hydrochloride can effectively reduce inflammation and oxidative damage in lung tissue, alleviate pulmonary edema and histopathological changes, and reduce pulmonary dysfunction and pulmonary fibrosis. Thus, it can be used to treat idiopathic pulmonary fibrosis. Further, it can be used to prepare a medicament for treating idiopathic pulmonary fibrosis.

### Preparation Examples of the Compound Dry Powder Inhalant

The B-90 nano spray dryer was used to produce the compound dry powder inhalant. PBS solution with different mass content of baicalin, amiloride hydrochloride and L-leucine was respectively formulated as feed solution. The ratio of each ingredient of feed solution in each example was shown in Table 1 below, and each feed solution was spray dried under the same parameters: an inlet temperature of 80° C., an air flow of 100 L·min⁻¹, a pump speed of 23%, a spray rate of 60%, and an internal pressure of 34 mbar. After spray drying, the DPI dry powder was collected in a dry environment at room temperature, weighed, sealed and stored in a desiccator at room temperature.

TABLE 1

| | | Ambroxol | | |
|---|---|---|---|---|
| | Baicalin | hydrochloride | L-leu | Ratio of L-leu |
| Example | (mg/ml) | (mg/ml) | (mg/ml) | content |
| 1 | 1 | 1 | 0 | 0% |
| 2 | 0.90 | 0.90 | 0.2 | 10% |
| 3 | 0.85 | 0.85 | 0.3 | 15% |
| 4 | 0.80 | 0.80 | 0.4 | 20% |
| 5 | 0.75 | 0.75 | 0.5 | 25% |
| 6 | 0.60 | 0.60 | 0.8 | 40% |
| 7 | 0.50 | 0.50 | 1 | 50% |

### Quality Evaluation of Compound Dry Powder Inhalant

### Ingredient Content Determination

About 10 mg of dry powder of different L-leu formulations prepared in Examples 1-7 was respectively weighed, placed in a 25 mL volumetric flask and diluted to the scale with methanol, shaken evenly, and used as the test sample. An analysis was carried out by high performance liquid chromatography to determine the content, and the amount of drug loading in different ratios of BA/AH-DPI dry powder was calculated. The drug loadings in different ratios of BA/AH-DPI dry powder in Examples 1-7 were obtained as shown in Table 2.

Preparation of BA control stock solution: 8.46 mg of BA control was precisely weighed, added into a 25 mL volumetric flask, dissolved in methanol, diluted to the scale, shaken evenly to obtain the control solution at a concentration of 338.4 μg/mL and stored at 4° C. under refrigeration.

Preparation of AH control stock solution: 8.73 mg of AH control was precisely weighed, added into a 25 mL volumetric flask, dissolved in methanol, diluted to the scale, shaken evenly to obtain the control solution at a concentration of 349.2 μg/mL and stored at 4° C. under refrigeration.

Preparation of the standard solution: 2 mL of BA and AH control stock solution was precisely weighed, added into a 10 mL volumetric flask, diluted to the scale with methanol solution, shaken evenly and stored at 4° C. under refrigeration. A standard solution containing 67.68 μg/ml BA and 69.84 μg/ml AH was prepared.

The calculation was as follows:

Drug loading=(peak area of the test sample/peak area of the standard)*concentration of the standard*25 ml/weighed mass of dry powder

TABLE 2
Contents of BA and AH in BA/AH-DPI dry powder with
different ratio (Mean ± SD, n = 3)

| Example | Ratio of L-leu content | BA (mg/mg) | AH (g/mg) |
|---|---|---|---|
| 1 | 0% | 0.35 ± 0.0003 | 0.32 ± 0.0036 |
| 2 | 10% | 0.36 ± 0.0028 | 0.33 ± 0.0037 |
| 3 | 15% | 0.33 ± 0.0015 | 0.31 ± 0.00018 |
| 4 | 20% | 0.3 ± 0.0029 | 0.3 ± 0.0025 |
| 5 | 25% | 0.26 ± 0.0015 | 0.25 ± 0.0013 |
| 6 | 40% | 0.22 ± 0.00038 | 0.21 ± 0.00038 |
| 7 | 50% | 0.19 ± 0.0022 | 0.2 ± 0.0021 |

As can be seen from the table, the dose range satisfied the dosing requirements. In addition, since the present application used PBS as the solvent, a certain amount of phosphate would present in the dry powder after spray drying. The content of phosphate in the BA/AH-DPI dry powder prepared in examples of the present application was about 15%-35%.

### Yield Determination

Yield (%)=mass of spray-dried powder−mass of spray-dried PBS powder/total mass

The mass of spray-dried powder: the mass of DPI dry powder prepared in 500 mL PBS with different formulation ratio according to the method of Examples 1-7.

The mass of spray-dried PBS dry powder: the mass obtained after 500 mL PBS solution was spray-dried;

Total mass: the sum of the mass of BA, AH, L-leucine added and the mass of each ingredient added in the preparation of 500 mL PBS solution.

The results of the yield of BA/AH-DPI prepared by spray drying were shown in Table 3 below. The yield of DPI particles with different formulation ratios was from 75% to 85%, which satisfied the requirements of DPI fine powder.

TABLE 3
Table of yield of spray-dried powder
with different formulation ratio

| Different L-leu | | | | | | | |
|---|---|---|---|---|---|---|---|
| ratios | 0% | 10% | 15% | 20% | 25% | 40% | 50% |
| Yield/% | 78.70 | 81.08 | 84.22 | 83.10 | 83.21 | 82.18 | 82.4 |

### Moisture Determination

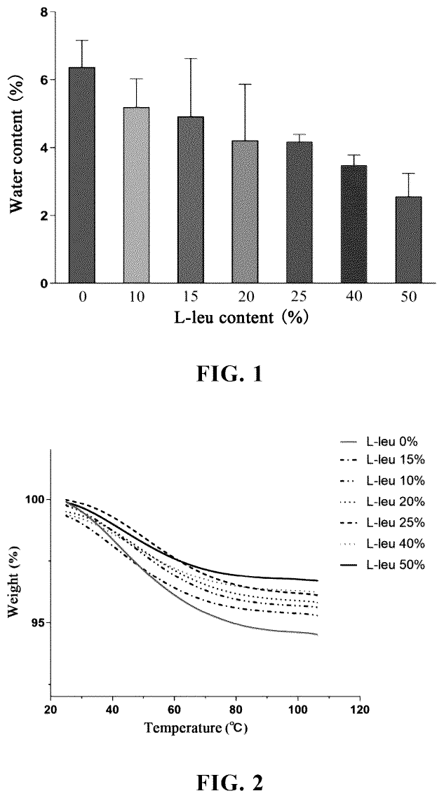

The moisture in BA/AH-DPI dry powder with different ratios in Examples 1-7 was measured by thermogravimetric analyzer (TGA), respectively, and the moisture in the fine powder was calculated based on the total amount of fine powder loss. The measured results of the moisture in BA/AH-DPI dry powder samples with different formulation ratios were shown in Table 4. The moisture trend charts were shown in FIG. 1 and FIG. 2.

TABLE 4
Moisture data for formulations with different L-leu ratios
(Mean ± SD, n = 3)

| Different formulation ratio | Water (%) |
|---|---|
| L-leu 0% | 6.37 ± 0.79 |
| L-leu 10% | 5.19 ± 0.85 |
| L-leu 15% | 4.91 ± 1.71 |
| L-leu 20% | 4.21 ± 1.65 |
| L-leu 25% | 4.17 ± 0.22 |
| L-leu 40% | 3.49 ± 0.30 |
| L-leu 50% | 2.56 ± 0.68 |

As can be seen from Table 4, the measured moisture content in BA/AH-DPI particles with different formulation ratios was less than 7.0%. From FIG. 1 and FIG. 2, it could be concluded that the moisture content gradually decreased with the addition of L-leu, indicating that the particles obtained by spray drying with L-leu could reduce the moisture content to prevent moisture effects.

### Observation of Particle Morphology

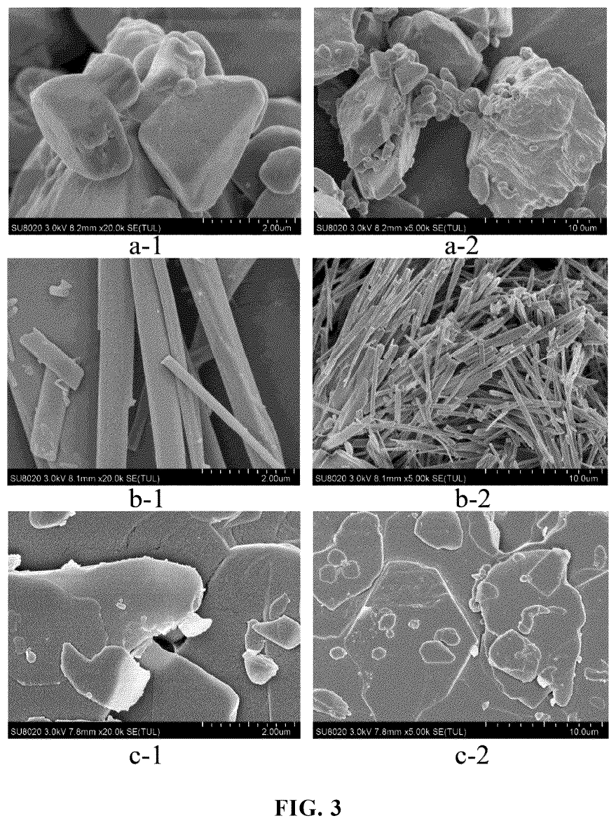

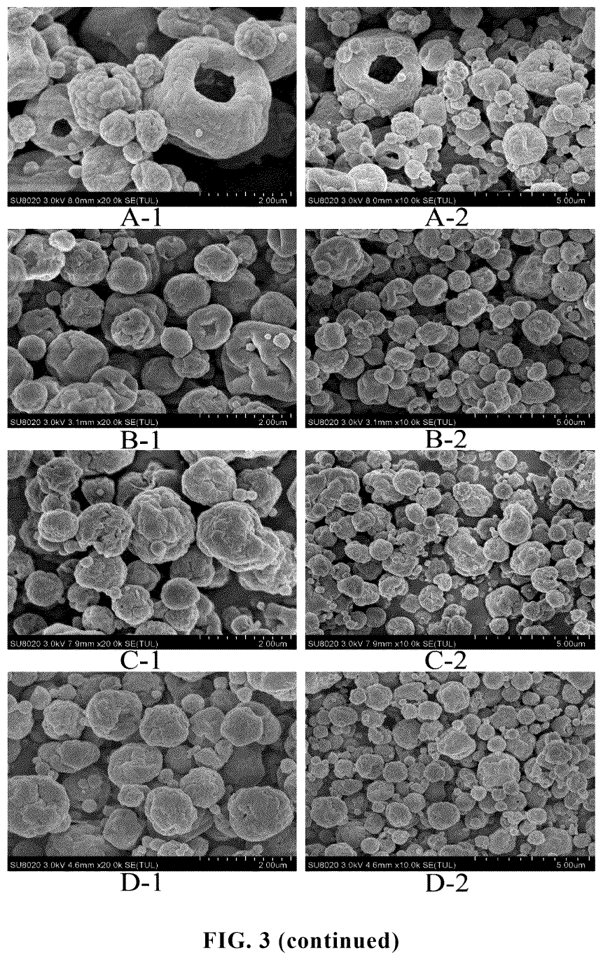

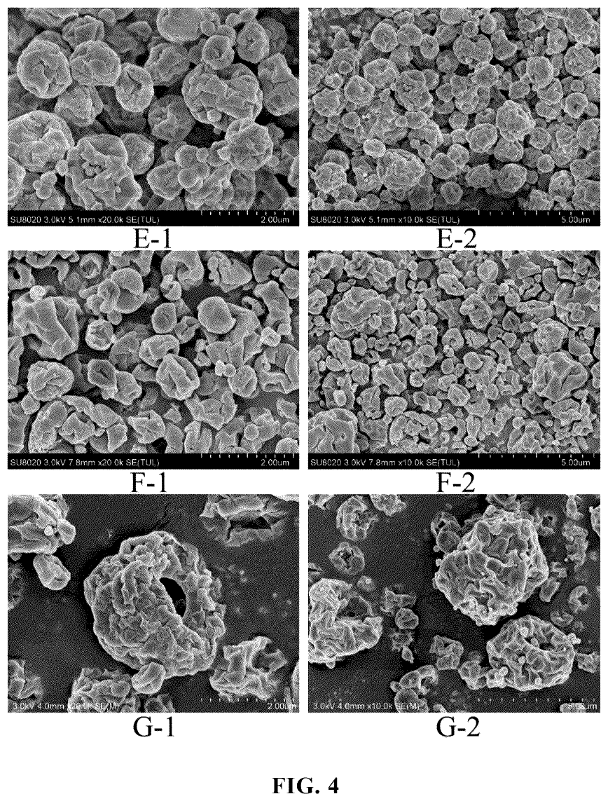

Scanning electron microscopy (SEM) was used to examine the surface morphology of BA, AH, L-Leu raw materials and BA/AH-DPI dry powders with different ratios, respectively. The sample powders were fixed uniformly on conductive adhesive tape, sprayed on an ion sputtering equipment for 30 s, and then placed on SEM to observe and acquire images with an image acquisition voltage of 10 kV. The SEM image results of BA, AH, and L-leu raw materials were shown in FIG. 3, in which figures a-1 and a-2 were the SEM images of BA at different magnifications, figures b-1 and b-2 were the SEM images of AH at different magnifications, and figures c-1 and c-2 were the SEM images of L-leu at different magnifications. The SEM image results of fine particles in BA/AH-DPI dry powders with different formulation ratios in Examples 1-7 were shown in FIG. 4, in which figures A-1 and A-2 were the SEM images of BA/AH-DPI fine particles with 0% L-leu content at different magnifications, and figures B-G were the SEM images of BA/AH-DPI fine particles with 10%, 15%, 20%, 25%, 40% and 50% L-leu content at different magnifications, respectively.

From the results in FIG. 3, it was clear that BA, AH and L-leu raw materials had irregular, strip-shaped, non-spherical morphology, whereas in FIG. 4, BA/AH-DPI dry powders with different ratios showed monodisperse particle size range, irregular folded flocculation, rough surface and pleated morphology of spherical morphological particles, which was consistent with that in the references. It could also be seen in FIG. 4 that the surface folded morphology of BA/AH-DPI derived from L-leu content from 10% to 40% was better and in uniform spherical size.

Without limited by any theory, the inventors analyzed and found that due to relatively high Péclet number (Pe) of L-leu, a hydrophobic layer was formed on the surface of the DPI fine particles obtained by spray drying, leading to the formation of corrugated fine particles, increasing the surface roughness of BA/AH-DPI fine particles and reducing the surface energy of the fine particles, which was the result of the enrichment of L-leu on the surface of the fine particles.

### Particle Size Distribution Test

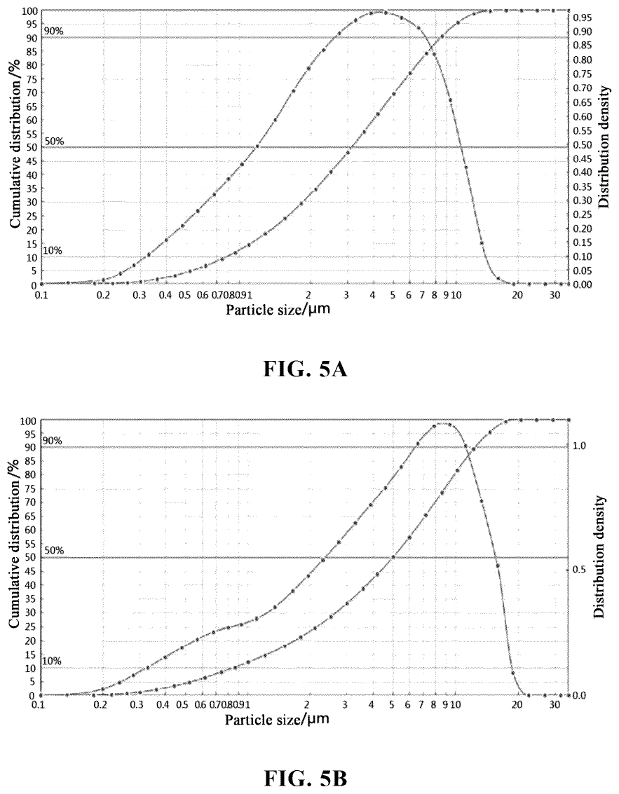

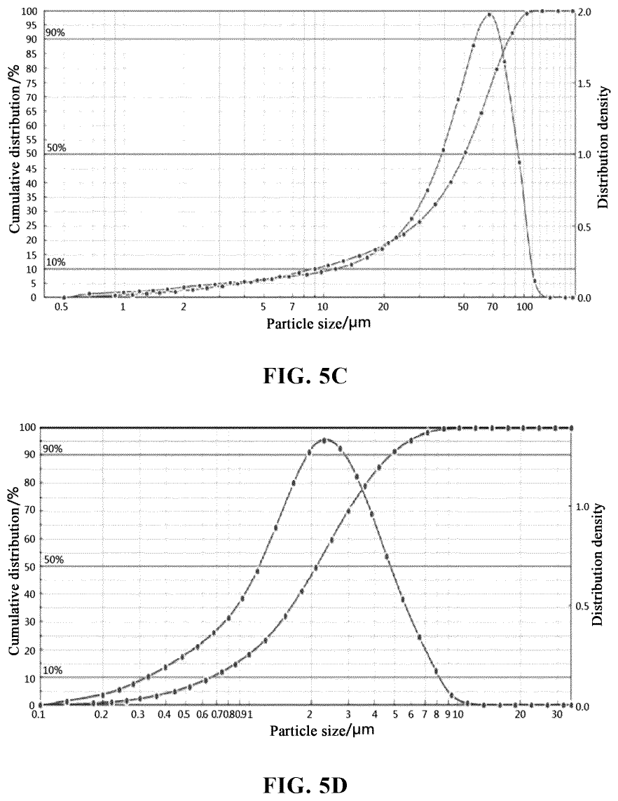

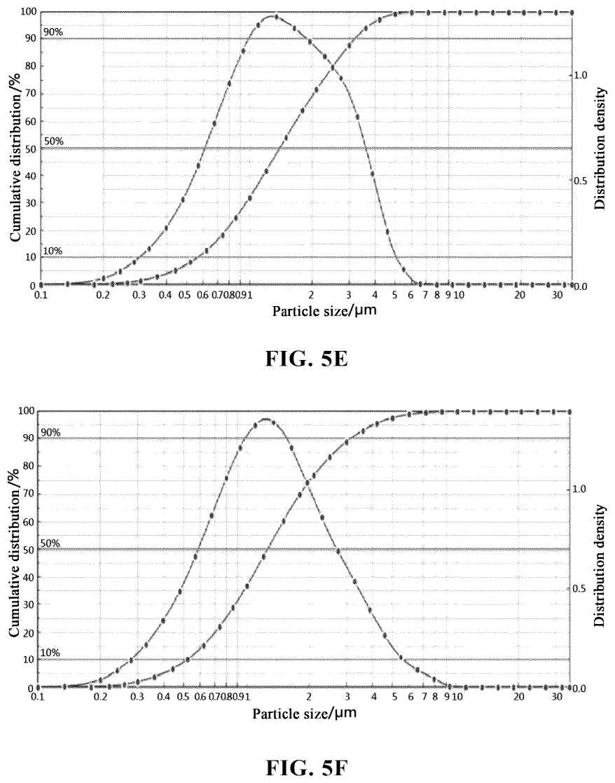

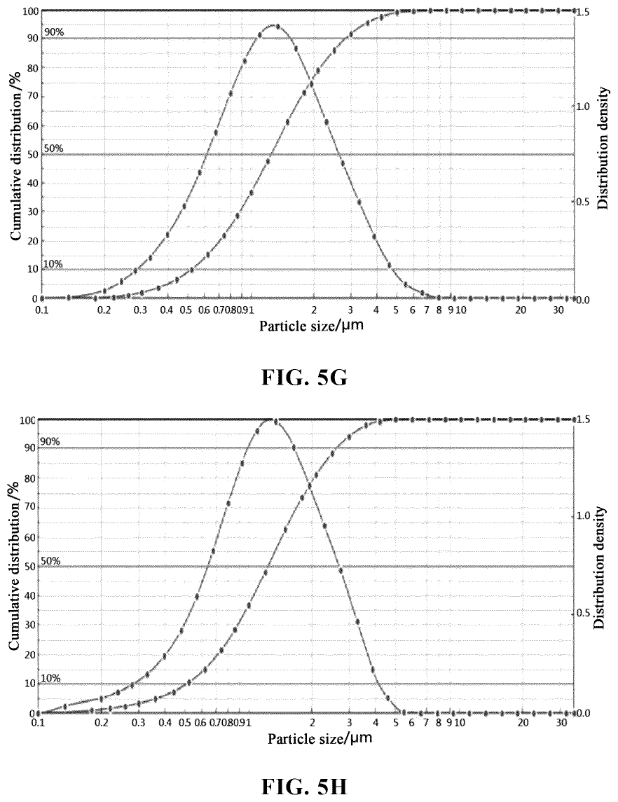

The particle size distribution of DPI dry powder was measured by dry dispersion method using laser particle size analyzer, and Dv10, Dv50, Dv90, VMD (Volume median diameter) and SMD (Sauter mean diameter) were determined, respectively. The results were shown in Table 5. The results of the particle size distribution curves obtained by plotting VMD and SMD were shown in FIG. 5A-FIG. 5J, in which FIG. 5A showed BA; FIG. 5B showed AH; FIG. 5C showed L-leu raw material; FIG. 5D-FIG. 5J showed BA/AH-DPI with L-leu contents of 0%, 10%, 15%, 20%, 25%, 40% and 50% (Examples 1-7) in turn.

TABLE 5
Measured results of particle size of BA/AH-DPI dry powder with different ratios

| particulate | D10/μm | D50/μm | D90/μm | <3 μm/% | <5 μm/% | VMD/μm | SMD/μm |
|---|---|---|---|---|---|---|---|
| L-leu | 9.39 | 50.97 | 84.70 | 3.80 | 6.08 | 49.54 | 16.64 |
| BA | 0.88 | 5.18 | 12.61 | 31.81 | 48.66 | 6.05 | 2.23 |
| AH | 0.77 | 3.07 | 8.43 | 49.10 | 70.30 | 3.93 | 1.79 |
| L-leu 0% | 0.59 | 2.01 | 4.67 | 71.94 | 92.15 | 2.38 | 1.26 |
| L-leu 10% | 0.55 | 1.41 | 3.35 | 86.40 | 98.77 | 1.71 | 1.08 |
| L-leu 15% | 0.53 | 1.24 | 2.85 | 91.66 | 99.23 | 1.50 | 1.00 |
| L-leu 20% | 0.53 | 1.38 | 2.80 | 92.46 | 99.55 | 1.52 | 0.98 |
| L Jeu 25% | 0.51 | 1.25 | 2.68 | 93.74 | 99.86 | 1.44 | 0.93 |
| L-leu 40% | 0.52 | 1.26 | 2.90 | 91.11 | 99.01 | 1.55 | 0.96 |
| L-leu 50% | 0.56 | 1.48 | 2.94 | 90.94 | 99.51 | 1.65 | 1.05 |

From the results in Table 5 and FIG. 5A-FIG. 5J, it could be seen that the particle size of active pharmaceutical ingredients was large, while the particle size of fine particle of each formulation after spray drying obviously satisfied the requirements of inhalation formulation. The particle size of Dv90 was below 5 μm, and the Dv90 of BA/AH-DPI was below 3 μm when the percentage of L-leu content was 10%-40%.

### Differential Scanning Calorimetry (DSC) Test

BA, AH, L-leu, and the physical mixture of the three prepared in a mass ratio of 2:2:1 were respectively precisely weighed with 5 mg of BA/AH-DPI dry powder (containing 20% L-leu) of Example 4, pressed in an aluminum sample tray, placed in the sample chamber of DSC, together with a blank reference tray. A dry N₂ environment (30 mL/min) was maintained. After the system was stable, the sample was detected in the temperature range from 30° C. to 350° C. at a heating rate of 10° C./min. The endothermic and exothermic curves of the sample were plotted.

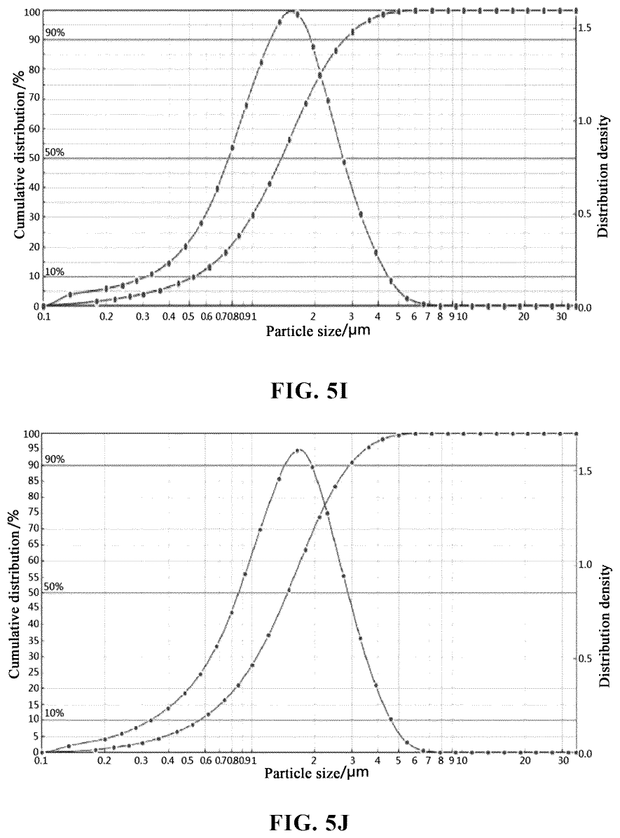

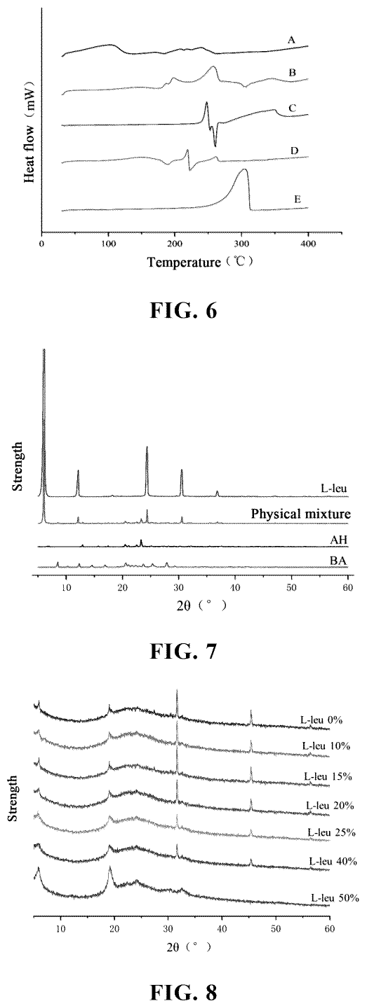

The DSC profiles of BA, AH, L-leu, the physical mixture of the three and BA/AH-DPI dry powder were shown in FIG. 6. From FIG. 6, it could he seen that BA (curve C) had an endothermic melting peak at 216.49° C., AH (curve D) had an endothermic melting peak at 246.26° C., L-leu (curve E) had an endothermic melting peak at 301.28° C.; while the physical mixture of the three (curve B) had an endothermic peak at 196.45° C., and it was assumed that the melting temperature of BA decreased under the influence of the excipient of L-leu. Meanwhile, in the physical mixture of the three, the endothermic peak of AH could be seen at 255.19° C. and the exothermic peak of L-leu could be seen at 302.26° C. In DPI (curve A), a small endothermic peak of melting appeared at 105.51° C. and 218.19° C., while the melting peak of BA weakened and the endothermic melting peaks of AH and L-leu disappeared, and no characteristic peaks of BA and AH appeared in the figure, indicating the transformation from the crystalline form of BA and AH to the amorphous form of DPI. Without limited to any theory, the inventors suggested that this might be the result that BA and AH were encapsulated in L-leu in molecular form after the formation of BA/AH-DPI dry powder, and transformed from the crystalline form to the amorphous form.

### X-Ray Diffraction (XRD) Analysis

BA, AH, L-leu, the physical mixture of the three (BA, AH, and L-leu), and an appropriate amount of BA/AH-DPI dry powder with different ratios of Examples 1-7 were respectively weighed, ground into a uniform powder, and the powder was made into a sample test piece with flat surface. The XRD curves were recorded by the Ni-filtered Cu—Kα radiation source, and the following detection conditions were set: voltage of 40 kV, 200 mA, scan range of 5°-60° (2θ), scan speed of 4°/min, and sampling time of 1 s. The XRD plots were plotted separately. The XRD plots of BA, AH, L-leu and the physical mixtures of the three were shown in FIG. 7. The XRD results of BA/AH-DPI dry powder with different ratios of Examples 1-7 were shown in FIG. 8.

It could be seen from FIG. 7 that BA, AH, L-leu and the physical mixture of the three were all in crystalline form, in which BA had strong diffraction peaks at 8.52°, 10.28°, 12.32°, 14.60°, 16.90°, 20.59°, 23.70°, 25.32°, 27.90° and 29.34°, AH also had strong diffraction peaks at 12.91°, 15.71°, 17.47°, 20.45°, 22.50°, 23.28° and 25.09°, and L-leu also had strong diffraction peaks at 6.13°, 12.15°, 24.37°, 30.53° and 36.82°. The physical mixture of the three also exhibited the same diffraction peaks, with strong diffraction peaks of crystalline features at 6.09°, 8.58°, 12.15°, 12.95°, 15.75°, 16.96°, 17.51°, 20.49°, 22.54°, 23.32°, 24.37°, 25.09°, 30.57° and 36.88°, suggesting that the drugs were all crystalline, and the crystalline form did not change after mixing.

Additionally, the results in FIG. 8 showed that the BA/AH-DPI dry powder was in a non-crystalline state. Although BA/AH-DPI fine powder had small broad weak diffraction peaks at 19.14°, 31.65° and 45.39°, the intensities of the diffraction peaks decreased with the addition of L-leu, until they disappeared and exhibited irregular electromagnetic radiation diffraction, indicating that the powder was mainly amorphous. Further, it was indicated that the fine powder obtained after spray drying changed from crystalline form to amorphous form, which confirmed the DSC results.

### In Vitro Aerodynamic Analysis

DPI deposition in vitro is usually measured in the art under ambient temperature and low relative humidity conditions to simulate the deposition of DPI in lungs.

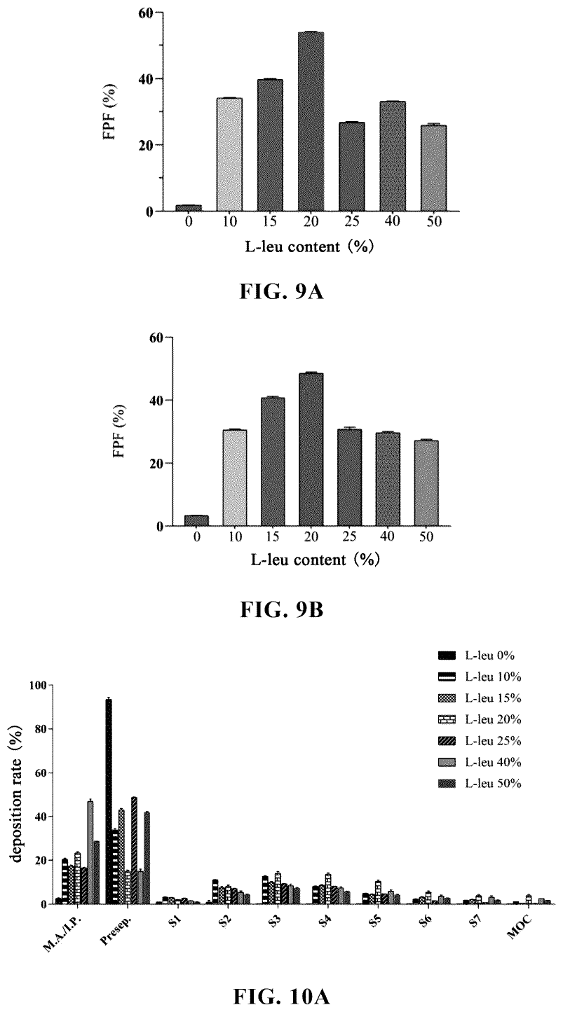

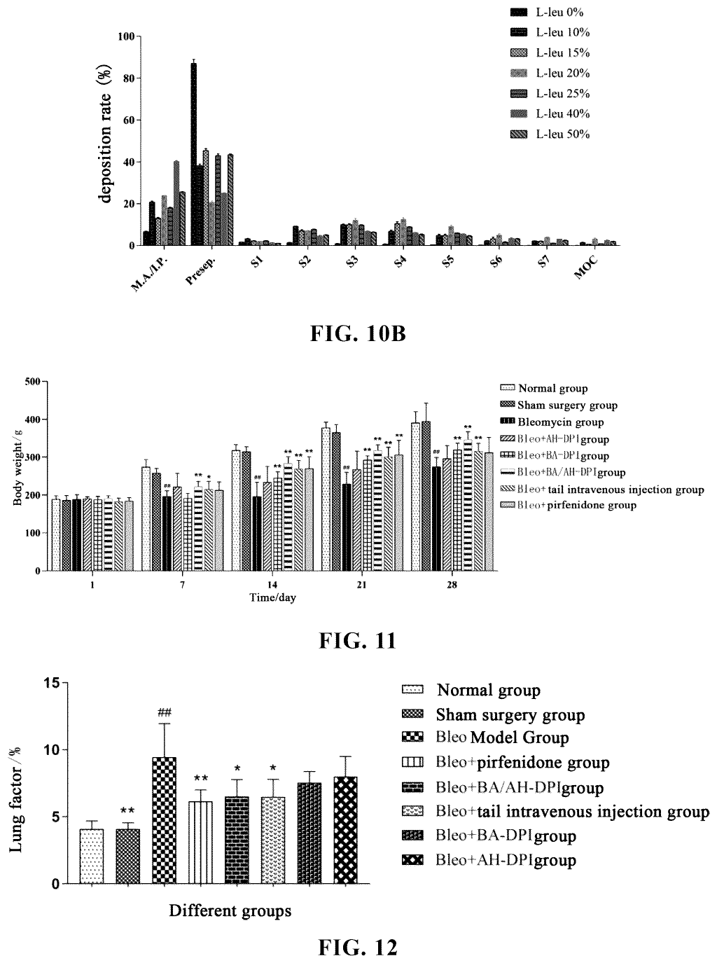

The present application used next generation pharmaceutical impactor (NGI) to determine the in vitro deposition properties of the BA/AH-DPI dry powder of Examples 1-7. The components of the instrument were connected sequentially, including the hinged lid of the NGI, the pre-separator (Presep.), the artificial throat (I.P.) and the inlet adapter (M.A.), and an ethanol solution containing a certain amount of benzyl and glycerol was added to the surface of each collection cup and left to evaporate for 0.5 h to avoid particle rebound or re-entrainment. BA/AH-DPI fine particles were added in excess to the Easyhaler multi-dose powder inhalant and tested (inhalation time: 4 s, inhalation rate: 60 L/min). After nebulization, a pre-determined volume of methanol was used for uniform and sufficient rinsing to dissolve the drug-containing fine powders in all phases, which were collected. The collected solutions were analyzed by HPLC according to the following chromatographic conditions and the gradient elution conditions shown in Table 6. Parameters of aerosol properties were calculated by CITDAS® software: fine particle fraction (FPF), mass median aerodynamic diameter (MMAD) and geometric standard deviation (GSD), and the results were shown in Table 7. The FPF measurements of BA and AH in BA/AH-DPI dry powder with different L-leu ratios were shown in FIG. 9A and FIG. 9B. The deposition of BA and AH in each layer of NGI was shown in FIG. 10A and FIG. 10B, respectively.

Liquid chromatographic conditions: the column was Agilent ZORBAX Eclipse Plus C18 (4.6×250 mm, 5 μm), mobile phase: methanol-0.1% aqueous phosphoric acid; detection wavelength: 248 nm; flow rate: 1.0 mL/min; column temperature: 30° C.; injection volume: 10 μL.

TABLE 6
Elution gradient conditions for BA/AH content determination

| Time (min) | Methanol (%) | 0.1% aqueous phosphoric acid (%) |
|---|---|---|
| 0 | 40 | 60 |
| 1 | 55 | 45 |
| 9 | 65 | 35 |
| 10 | 50 | 50 |
| 11 | 50 | 50 |

TABLE 7
Results of in vitro deposition properties of BA/AH-DPI

| | Different | | | | | | |
|---|---|---|---|---|---|---|---|
| | L-leu | BA | | | AH | | |
| Example | ratios | FPF/% | MMAD/μm | GSD | FPF/% | MMAD/μm | GSD |
| 1 | 0% | 1.84 ± 0.020 | 5.01 ± 0.13 | 6.2 ± 0.12 | 3.26 ± 0.16 | 4.76 ± 0.13 | 3.48 ± 0.24 |
| 2 | 10% | 34.17 ± 0.13 | 3.28 ± 0.022 | 1.86 ± 0.015 | 30.59 ± 0.28 | 3.11 ± 0.028 | 2.01 ± 0.014 |
| 3 | 15% | 39.75 ± 0.28 | 3.38 ± 0.01 | 2.03 ± 0.002 | 40.71 ± 0.51 | 2.18 ± 0.01 | 2.14 ± 0.008 |
| 4 | 20% | 53.95 ± 0.17 | 2.15 ± 0.006 | 2.09 ± 0.005 | 48.45 ± 0.16 | 2.12 ± 0.005 | 2.15 ± 0.021 |
| 5 | 25% | 26.78 ± 0.17 | 3.07 ± 0.01 | 1.96 ± 0.006 | 30.79 ± 0.66 | 2.89 ± 0.015 | 1.95 ± 0.006 |
| 6 | 40% | 33.13 ± 0.15 | 2.2 ± 0.01 | 2.13 ± 0.003 | 29.66 ± 0.48 | 2 ± 0.035 | 2.37 ± 0.036 |
| 7 | 50% | 25.89 ± 0.54 | 2.29 ± 0.015 | 2.12 ± 0.006 | 27.11 ± 0.43 | 2.27 ± 0.080 | 2.07 ± 0.036 |

From the results in Table 7 together with FIG. 9A and FIG. 9B, it could be seen that the FPF values of both BA and AH in the formulation with L-leu were higher compared to DPI without L-leu; with the increase of L-leu content, the FPF of dry powder gradually increased and reached the highest at 15-25%, especially at 20%, indicating that the best in vitro deposition properties of BA/AH-DPI dry powder were achieved at this time, and with further increase of L-leu content, the FPF values decreased instead. It was indicated that the moderate addition of L-leu carrier to DPI could improve the in vitro deposition properties such as FPF of DPI dry powder to some extent.

The in vitro aerodynamic study showed that the addition of L-leu significantly increased the FPF of the fine powder (53.95±0.17%) relative to that that of fine powder without the addition of L-leu, which had an important role in improving the dispersibility of BA/AH-DPI, indicating that the moderate addition of L-leu resulted in excellent aerosol properties and physical nebulization stability of the BA/AH-DPI of the present application.

The above experimental results showed that the in vitro aerodynamic measurements were optimal when the percentage of L-leu was 15-25%. Subsequently, the optimal formulation with 20% of L-leu was selected to further investigate the in vivo efficacy of BA/AH-DPI.

### Pharmacodynamic Studies

### Laboratory Animals and Drugs

The study was approved by the Medical Ethics Committee of Tianjin University of Traditional Chinese Medicine and conducted at the Animal Experimentation Center, and the animals used in the project were carried out in strict accordance with the approved research protocol.

Bleomycin (Bloe): Zhejiang Haizheng Pharmaceutical Co., Ltd; bleomycin was dissolved in saline solution to prepare a bleomycin solution with a concentration of 12.5 mg/mL for tracheal intubation administration.

BA-DPI: BA-DPI was prepared by dissolving 400 mg BA and 200 mg L-leucine in 500 mL PBS under the same spray drying conditions as in Example 4 (the BA content in the dry powder was 0.638 mg/mg).

AH-DPI: AH-DPI was prepared by dissolving 400 mg AH and 200 mg L-leucine in 500 mL PBS under the same spray drying conditions as in Example 4 (AH content in dry powder was 0.571 mg/mg).

BA/AH-DPI: The BA/AH-DPI dry powder of Example 4 of the present application was used, in which in the following pharmacodynamic studies and results, BA/AH-DPI referred to BA/AH-DPI of Example 4 if it was not otherwise specified.

Tail intravenous injection formulation solution: The BA/AH-DPI dry powder with 20% L-Leu content of Example 4 was dissolved in physiological saline to prepare tail intravenous injection formulation solution with a concentration of 3 mg/ml.

Pirfenidone: Manufacturer Beijing Kantini Pharmaceutical Co., Ltd, specification 100 mg, Cat No. National Medicine Permit No. H20133376. Pirfenidone was a clinical drug commonly used in the treatment of pulmonary fibrosis, and pifinidone was used as a positive control in the present application. Pirfenidone was dissolved in physiological saline solution to prepare pirfenidone solution with a concentration of 11.6 mg/ml for gavage administration.

### Experimental Animal Grouping and Processing

48 SPF healthy SD male rats (weight of 180-220 g) were randomly divided into 8 groups, 6 rats for each group: (1) normal group (control group): normal rearing in the animal center; (2) sham group: this group was given the same dose of air by pulmonary administration; (3) Bleo model group: 100 μL of Bleo solution with a concentration of 12.5 mg/mL was administered through the tracheal intubation, and the rats were rotated for 5 min after drug administration for use, referred to as the Bleo group or the model group in the present application; (4) Bleo+BA-DPI group: the same modeling method as the Bleo model group was used, and BA-DPI was administered through the tracheal intubation via DP-4 rat dry powder lung drug delivery device (manufacturer: Beijing YSKD Biotechnology Co.) at a dose of 9 mg/kg, 3 mg per day for 28 days; (5) Bleo+AH-DPI group (same method as 4); (6) Bleo+BA/AH-DPI group (same method as 4); (7) Bleo+tail intravenous injection group: the formulation solution was administered by tail intravenous injection at a concentration of 3 mg/ml, 9 mg/kg, for a total of 28 days, starting from the second day after the modeling; (8) Bleo+pirfenidone group: 2 ml per day at a concentration of 11.6 mg/ml was administered by gavage starting from the second day after successful modeling for 28 days.

Lung DPI dry powder administration operation: DPI powder was weighed precisely and placed in the storage room of the drug delivery device, the DP4 dry powder delivery device was installed, connected to the syringe with 1.5-2 ml of air, the rat was laid on the tracheal intubation platform after anesthesia, the rat laryngoscope was used to expose the tracheal opening, when the tracheal opening was open, the nozzle of the drug delivery device was immediately inserted through the tracheal opening to the bronchial fork, and the air was immediately pushed into the drug delivery device to push DPI fine powder into the rat's lung. The rat in sham group was pushed with 1.5-2 ml of air (not including the drug) as described above.

### Data Analysis

Experimental results were expressed as Mean±SD. Differences between groups were analyzed by one-way ANOVA and Tukey's multiple comparison test. p<0.05 indicated a significant difference. All data calculations and analyses were performed by using GraphPad Prism 8.0.

### Analysis of Body Weight Changes in Rats

Rats were observed daily for respiration, activity, feeding and body weight. Bleo modeling was considered as the first day, and the body weight of each group was recorded on days 1, 7, 14, 21 and 28, respectively. The results were statistically analyzed as shown in FIG. 11. The rats in the normal and sham groups were reared normally, in good mental condition, with normal diet, and their body weight continued to increase. As seen in FIG. 11, there was no difference between the weight gain of rats in the sham group and the normal group. The body weight of rats in the Bleo group remained basically unchanged within 14 days, while the rats in the Bleo+AH-DPI group were in poor mental condition and slightly depressed after modeling, with reduced diet and water intake, lazy movement and easy aggregation, and dull complexion of both ears and extremities, and their body weights gradually increased after day 7, but the increase in body weight was not obvious compared with that of the Bleo group. The rats in Bleo+BA-DPI and Bleo+BA/AH-DPI groups had a slight increase in body weight and less activity in the first 7 days, and after 7 days the rats were more active, drinking and eating normally and gaining weight rapidly. The rats in Bleo+tail intravenous injection group and Bleo+pirfenidone group showed an increase in body weight compared with the Bleo+BA/AH-DPI group, and the animal condition was slightly improved, but still worse compared with that in Bleo+BA/AH-DPI group. Among them, the body weight of rats in the normal group, Bleo+BA/AH tail intravenous injection group and Bleo+BA/AH-DPI group was significantly higher than that in the Bleo group from day 7 (p<0.01 or p<0.05), and the body weight of rats in the Bleo+pirfenidone group was significantly different from that in the Bleo group from day 14 (p<0.01). After 28 days of tail intravenous injection, all of the rats had some discomfort in their condition and some degree of tail desquamation. In conclusion, BA/AH-DPI significantly improved the weight loss in rats having Bleo-induced pulmonary fibrosis.

### Pulmonary Function Index Assay

Various respiratory indices, including inspiratory duration (Ti), expiratory duration (Te), maximal inspiratory flow (PIF), maximal expiratory flow (PEF), tidal volume (TV), relaxation time (RT), and expiratory volume (EV), in freely moving rats were measured by EMKA pulmonary function detector at the completion of modeling on day 28. The results were shown in Table 8.

TABLE 8
Pulmonary function index data of different groups of rats

| Different | | | | | | | |
|---|---|---|---|---|---|---|---|
| groups | Ti (ms) | Te (ms) | PIF (ml/s) | PEF (ml/s) | TV (ml) | EV (ml) | RT (ms) |
| Bleo group | 154.36 ± 56.98** | 223.34 ± 61.05** | 8.89 ± 4.56** | 7.99 ± 5.61** | 1.15 ± 0.22** | 1.14 ± 0.19** | 126.96 ± 33.41** |
| Bleo + | 162.16 ± 36.37 | 227.11 ± 76.67 | 11.71 ± 3.34** | 11.13 ± 3.86** | 1.18 ± 0.21 | 1.18 ± 0.24 | 142.63 ± 57.44 |
| AH-DPI | | | | | | | |
| group | | | | | | | |
| Bleo + | 159.46 ± 42.68 | 243.13 ± 97.85 | 10.90 ± 5.26** | 11.43 ± 7.70** | 1.18 ± 0.25 | 1.19 ± 0.21 | 152.26 ± 77.88** |
| BA-DPI | | | | | | | |
| group | | | | | | | |
| Bleo + | 186.16 ± 61.68** | 282.32 ± 105.28** | 11.87 ± 6.37** | 10.42 ± 6.82** | 1.22 ± 0.29 | 1.22 ± 0.30 | 166.04 ± 64.77** |
| BA + AH | | | | | | | |
| tail | | | | | | | |
| intravenous | | | | | | | |
| injection | | | | | | | |
| group | | | | | | | |
| Bleo + | 190.07 ± 65.75** | 281.08 ± 136.72** | 12.43 ± 6.08** | 12.49 ± 9.35** | 1.24 ± 0.20** | 1.29 ± 0.21** | 163.53 ± 84.65** |
| BA/AH-DPI | | | | | | | |
| group | | | | | | | |
| Bleo + | 180.56 ± 49.34** | 265.48 ± 99.45** | 12.52 ± 4.68** | 10.76 ± 5.93** | 1.22 ± 0.30 | 1.23 ± 0.30* | 158.82 ± 63.19** |
| pirfenidone | | | | | | | |
| group | | | | | | | |
| Sham group | 223.11 ± 70.96** | 317.27 ± 111.37** | 13.11 ± 6.67** | 13.06 ± 9.69** | 1.34 ± 0.57** | 1.34 ± 0.55** | 196.18 ± 56.32** |
| Normal | 219.26 ± 43.13 | 318.23 ± 121.76 | 14.78 ± 10.08 | 15.13 ± 15.46 | 1.37 ± 0.24 | 1.37 ± 0.29 | 178.46 ± 92.72 |
| group | | | | | | | |

Compared with normal group,
*p < 0.05,
**p < 0.01;
compared with Bleo group,
*p < 0.05;
**p < 0.01

Pulmonary resistance and compliance were often considered to be the gold standard for assessing lung function. The data of lung function parameters were shown in Table 8. Compared with the normal group, Ti, Te, PIF, PEF, RT, TV, and EV values were lower in the lung function parameters of rats in the Bleo group (p<0.01, p<0.05). Compared with the Bleo group, PIF and PEF were more significantly elevated in the Bleo+AH-DPI group in the lung function parameters (p<0.01). The Bleo+BA-DPI group showed significant elevation in PIF, PEF, and RT in pulmonary function parameters. Bleo+BA/AH tail intravenous injection group, Bleo+BA/AH-DPI group and Bleo+pirfenidone group had higher Ti, Te, PIF, PEF, RT in pulmonary function parameters than Bleo group (p<0.01), and Bleo+BA/AH-DPI group had significant differences in parameters of TV and EV compared to Bleo group (p<0.01, p<0.05). The above results indicated that the BA/AH-DPI group of the present application had more significant improvement on lung function indices compared to the DPI administration of single BA or AH, and the administration by tail intravenous injection.

### Determination of Lung Factor in Rats

Pulmonary edema was a recognized indicator of pulmonary fibrosis, and the lung factor was one of the indicators of the degree of lung tissue damage, pulmonary edema and fibrosis. The higher of the value, the more severe the lung lesion. The following equation was used to calculate the lung factor and to perform statistical analysis.

The equation was as follows.

$$Lung factor = \frac{Lung wet weight ( g )}{Body weight ( kg )} \times 100 \%$$

After the 8 groups of rats (6 rats in each group) were killed, the alveolar lavage fluid of the rats was collected (for subsequent experiments). Lung tissues were extracted and weighed, and the lung factor values obtained were shown in Table 9 and FIG. 12. From the results, it could be seen that the lung factors of both the normal and sham groups were between 4 and 5; and the lung factor of the Bleo group was greater than 8, indicating that the index satisfied the requirements of the pulmonary fibrosis model. The lung factors of Bleo+AH-DPI and Bleo+BA-DPI groups were greater than 7, and those of rats in Bleo+BA/AH-DPI, Bleo+BA/AH tail intravenous injection and Bleo+pirfenidone groups were all in the range of 6-7, indicating that the drug combination of BA/AH had better effects on improving pulmonary edema, compared with the single drug.

TABLE 9
Data of lung tissue factors in each group of rats (n = 6)

| Group | Lung factor (Mean ± SD) |
|---|---|
| Normal group | 4.09 ± 0.61 |
| Sham group | 4.11 ± 0.45 |
| Bleo group | 9.45 ± 2.48 |
| Bleo + pirfenidone gavage group | 6.15 ± 0.84 |
| Bleo + BA/AH-DPI group | 6.44 ± 1.38 |
| Bleo + tail intravenous injection group | 6.47 ± 1.32 |
| Bleo + BA-DPI group | 7.51 ± 0.85 |
| Bleo + AH-DPI group | 7.98 ± 1.52 |

### Observation of HE Staining of Rat Lung Tissue

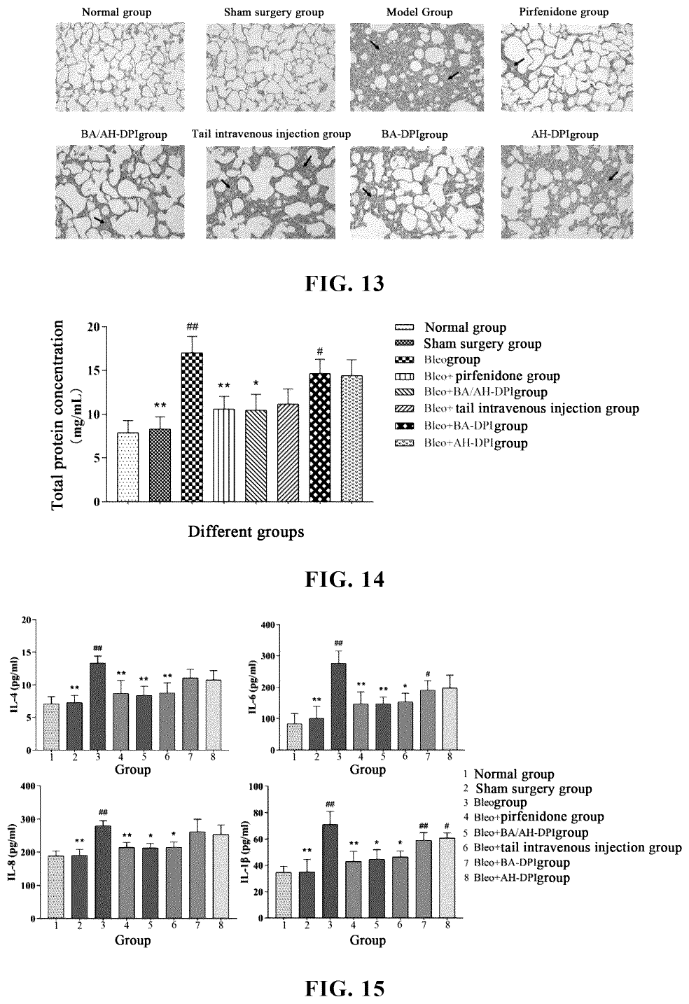

The weighed left lungs were stored in 10% neutral formalin fixative for 24 h, then dehydrated and embedded in paraffin. The sections were 5 μm in thickness, stained with hematoxylin and eosin (HE staining), dehydrated to transparent, and sealed with neutral gum, and then the specimens were observed under a microscope and photographed. The results were shown in FIG. 13.

As seen in FIG. 13, the normal and sham groups had fewer histopathological changes in lungs, with normal sections and thin alveolar septa; whereas the lung tissue of rats in the Bleo model group of showed severe fibrosis, exhibiting structural disorganization in a typical fibrotic appearance, including inflammatory cell infiltration and alveolar hemorrhage, the arrows in the figure indicating alveolar enlargement and alveolar interstitial thickening. Compared with the Bleo model group, the alveolar interstitial thickening was thinner in the Bleo+tail intravenous injection group, Bleo+pirfenidone group and Bleo+BA/AH-DPI group, with less fibrotic features and inflammatory infiltration. The pathological findings in the Bleo+BA-DPI and Bleo+AH-DPI groups were similar to those in the Bleo group, with slightly thinner alveolar interstitium than the Bleo group and inflammatory exudates, but there was no significant difference. This experiment illustrated that the BA/AH-DPI of the present application ameliorated the histopathological changes caused by Bleo.

### Analysis of Lung Bronchoalveolar Lavage Fluid (BALF)

The alveolar surface lining fluid obtained by using bronchoalveolar lavage was called bronchoalveolar lavage fluid (BALF), which was mainly used clinically for the detection of cytokines, oxidative stress-related enzymes and soluble substances for the determination, diagnosis, treatment, efficacy and improvement of the prognosis of lung-related diseases (such as lung inflammation, IPF, etc.).

The bronchoalveolar lavage (BAL) performed in this experiment was as follows: after sampling blood from the abdominal aorta of the rats, executing the rats, and dissecting the thoracic cavity to remove excess adipose tissue over the airway and to expose the trachea, inserting the lagging needle through the anterior end of the trachea so that the needle reached the right lung bronchus, fixing the needle both anteriorly and posteriorly, slowly instilling 2 ml of precooled saline into the lung, holding for 2 min, performing aspiration, repeating the operation three times, combining the three BALFs in a 10 ml EP tube and storing at low temperature. The recovery rate of lavage by this method was >80%.

The resulting BALF was centrifuged at 4° C. and 4000 r·min⁻¹ for 10 min to obtain the supernatant. The total protein content was determined by colorimetric method according to the instructions of Total Protein Determination Kit (Manufacturer: Nanjing Jiancheng Institute of Biological Engineering Co., Ltd; Cat No.: A045-4-2). The results of the total protein content measurement were shown in the following Table 10 and FIG. 14. The total protein content was about 8 mg/mL in both the normal group and the sham group, and there was no difference therebetween (p>0.05). Compared with the sham group, the total protein content was 17.04±1.87 mg/mL in the Bleo group, which was significantly increased (p<0.01). Compared with the Bleo group, the total protein content in BALF was lower in both the Bleo+pirfenidone group (p<0.01) and the Bleo+BA/AH-DPI group (p<0.05); whereas the total protein content decreased in the Bleo+tail intravenous injection group, the Bleo+AH-DPI group and the Bleo+BA-DPI group (p<0.05). The effect of the combination of the two drugs in reducing the total protein content was more significant compared with that of the single drug, and showed no significant difference from that of pirfenidone (p>0.05).

TABLE 10
Measurements of total protein in BALF (Mean ± SD, n = 6)

| | Total protein |
|---|---|
| Group | concentration (mg/mL) |
| Normal group | 7.86 ± 1.43 |
| Sham group | 8.33 ± 1.37 |
| Bleo group | 17.04 ± 1.87 |
| Bleo + pirfenidone group | 10.59 ± 1.47 |
| Bleo + BA/AH-DPI group | 10.46 ± 1.84 |
| Bleo + tail intravenous injection group | 11.17 ± 1.75 |
| Bleo + BA-DPI group | 14.62 ± 1.61 |
| Bleo + AH-DPI group | 14.39 ± 1.81 |

In order to verity whether BA/AH-DPI can regulate the inflammatory process by modulating the secretion of cytokines, the application also measured the concentrations of inflammatory factors IL-4, IL-6, IL-8, and IL-1β in BALF to determine the protective and therapeutic effects of BA/AH-DPI in the pathogenesis of Bleo-induced idiopathic pulmonary fibrosis.

The above BALF supernatant was measured by enzyme-linked immunosorbent assay (ELISA) to detect the concentration levels of inflammatory factors IL-4, IL-6, IL-8, IL-1β, as well as IFN-γ and TGF-β1. The specific experimental operations were performed according to the ELISA kit (IL-4, IL-6, IL-1β manufacturer: Wuhan Boster Biological Technology Co., Ltd; Cat No.: EK0406, EK0412, EK0393. IL-8 manufacturer: Shanghai Kexing Biotechnology Co., Ltd; Cat No.: F8655-A) instruction steps. The experimental results were shown in FIG. 15.

As shown in FIG. 15, the levels of inflammatory factors IL-4, IL-6, IL-8, and IL-1β concentrations in BALF of rats in the normal and sham groups were comparable (p>0.01). Compared with the normal group, the levels of IL-4, IL-6, IL-8 and IL-1β were significantly higher in the BALF of rats in the Bleo group (##p<0.01). Compared with the Bleo group, IL-4, IL-8 and IL-1β were significantly lower in the Bleo+pirfenidone group, Bleo+BA/AH-DPI group and Bleo+tail intravenous group (p<0.01 or p<0.05). Additionally, the concentration values of IL-4, IL-6, IL-8 and IL-1β in BALF of rats in Bleo+BA-DPI group and Bleo+AH-DPI group were reduced, all of which were not significantly different (p>0.05). The results showed that BA/AH-DPI was able to significantly reduce the level of inflammatory factors and had an inhibitory effect on the inflammatory response in Bleo-induced rats.

### Analysis of Hydroxyproline (Hyp) and Oxidative Stress Indicators in Lung Tissue Homogenates

The right lung was weighed and rinsed in physiological saline, and then blotted with filter paper, and subjected to lung tissue homogenization.

Oxidative stress played an important role in the process of Bleo-induced pulmonary fibrosis. The levels of MDA expression and SOD activity could reflect whether the oxidative and antioxidant effects of the body were in balance. Therefore, in the application, SOD was measured in conjunction with MDA in order to observe the role in pulmonary fibrosis. Hyp content was highest in collagen. The level of Hyp content in lung tissue homogenates showed the collagen metabolism in lung tissue and the degree of lung fibrosis.

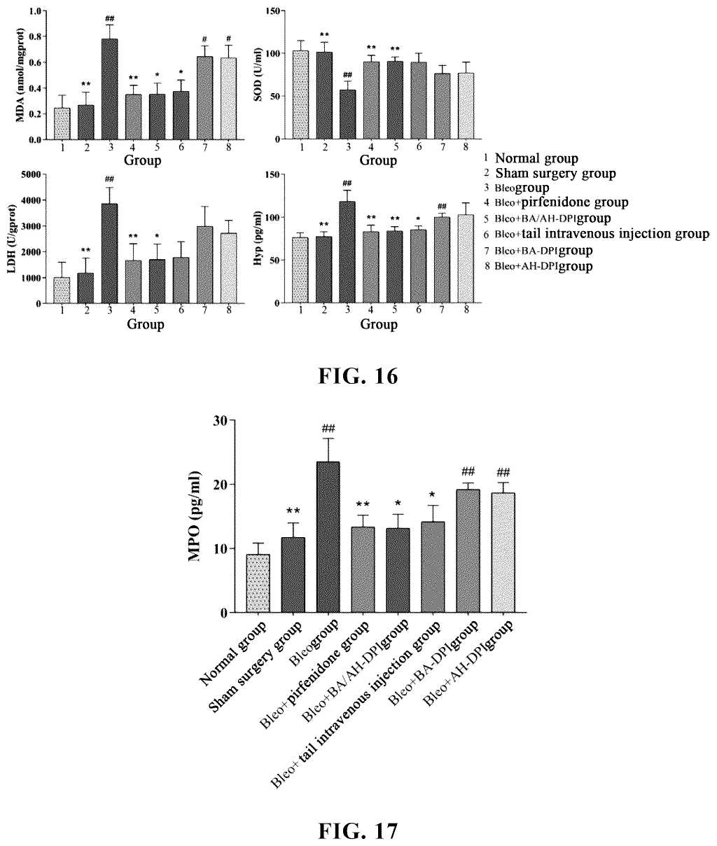

The application uses ELISA for the detection of Hyp level, in accordance with the ELISA kit (manufacturer: Shanghai Kexing Biotechnology Co., Ltd: Cat No.: F3609-A) instruction steps. The superoxide dismutase WST-1 method was used to detect the SOD concentration level. The thiobarbituric acid (TBA) method was used to detect the MDA concentration level. The microplate method was used to detect the lactate dehydrogenase (LDH) concentration level. The experimental operations were performed according to the SOD, MDA and LDH assay kits (SOD, MDA and LDH manufacturer: Nanjing Jiancheng Institute of Biological Engineering Co., Ltd; Cat No.: A001-3, A003-1, A020-2) instruction steps from the manufacturer for detection and analysis. The results of Hyp, SOD, MDA and LDH assays were shown in FIG. 16.

As shown in FIG. 16, compared to the normal group, after being induced by the administration of Bleo, the oxidative metabolites MDA and LDH were significantly increased (p<0.01) and SOD was significantly decreased (p<0.01) in rat lung tissues. Compared with the Bleo group, after 28 days of continuous administration, the MDA levels in the lung tissues of rats in the Bleo+pirfinidone, Bleo+BA/AH-DPI and Bleo+tail intravenous injection groups were significantly decreased (p<0.01, p<0.05), and the antioxidant indexes of SOD were significantly increased (p<0.01) and LDH content significantly decreased (p<0.01 or p<0.05) in the lung tissues of rats in the Bleo+pirfinidone and Bleo+BA/AH-DPI groups, while SOD and LDH in Bleo+tail intravenous infection group had some tendency of increasing and decreasing, but none of which were significantly different (p>0.05); and MDA and LDH levels in lung tissue of rats were decreased (p>0.05) and the SOD antioxidant index increased (p>0.05) in Bleo+BA-DPI group and Bleo+AH-DPI group. Bleo+BA-DPI and Bleo+AH-DPI groups had higher MDA levels (p<0.05) and lower SOD antioxidant indexes (p<0.05) in rat lung tissue relative to the normal group. This indicated that the BA/AH-DPI of the present application was effective in ameliorating oxidative damage in lung tissue of rats with pulmonary fibrosis.

The Hyp contents in the lung tissues of rats in the normal group and the sham group were 76.43 pg/ml and 77.57 pg/ml, respectively, and there was no significant difference therebetween. The Hyp content in the Bleo group was 118.33 pg/ml, which was significantly higher than that in the normal group and the sham group (p<0.01). Compared with the Bleo group, Hyp levels in the lung tissues of rats in the Bleo+pirfenidone group (content of 83.40 pg/ml), Bleo+BA/AH-DPI group (content of 84.06 pg/ml) and Bleo+tail intravenous injection group (content of 85.65 pg/ml) were significantly decreased (p<0.01, p<0.05) and close to the levels in the normal group and the sham group (as in FIG. 16). The Hyp content of lung tissue in the Bleo+AH-DPI group and the Bleo+BA-DPI group was 100.17 pg/ml and 102.97 pg/ml, respectively, which were significantly higher than those in the normal group and the sham group (p<0.05), but lower than those in the Bleo group. There were no significant differences between the three groups (p>0.05). This demonstrated that the BA/AH-DPI of the present application can improve pulmonary fibrosis in rats.

### Analysis of Myeloperoxidase Levels (MPO) in Serum

The blood was collected from rats via the abdominal aorta before execution. The whole blood was centrifuged at 4000 r·min⁻¹ for 10 min. The supernatant was extracted to obtain the serum. The serum was centrifuged at 4° C. and 4000 r·min⁻¹ for 10 min to obtain the supernatant for the determination of MPO content by colorimetric method. The experimental operation was performed according to the instructions from the manufacturer with the kit (manufacturer: Shanghai Kexing Biotechnology Co., Ltd; Cat No.: F3213-A) to detect the MPO level.

The MPO level in serum reflected the accumulation of neutrophils in lung tissue and was a reliable indicator of inflammatory cell infiltration in the lung. The measured results of MPO in serum of rats in the present application were shown in FIG. 17, which indicated that the MPO level in serum of rats was increased by Bleo induction. Compared with the normal group, the MPO level in serum were significantly higher in rats of the Bleo group, Bleo+BA-DPI group and Bleo+AH-DPI group (p<0.01). Compared with the Bleo group, the Bleo+pirfenidone, Bleo+BA/AH-DPI and Bleo+tail intravenous injection groups showed significantly inhibited MPO activity (p<0.01 or p<0.05); while the Bleo+BA-DPI and Bleo+AH-DPI groups showed slightly inhibited MPO activity, without significant difference (p>0.05). This indicated that the BA/AH-DPI of the present application was able to inhibit MPO activity and thus improve inflammatory cell infiltration in the lung.

Considering the above pharmacodynamic study results, the BA/AH-DPI of the present application can improve the lung function of rats with pulmonary fibrosis, and also had a good improvement effect on pulmonary edema and lung pathological changes. At the same time, the inventors also found that the BA/AH-DPI of the present application could improve lung inflammation and oxidative damage, and reduce the Hyp content in the lung, indicating that the BA/AH-DPI of the present application can improve the lung fibrosis symptoms. Additionally, the experimental data showed that the BA/AH-DPI of the present application has better therapeutic effect on pulmonary fibrosis compared with both the administration of DPI alone or BA/AH by tail intravenous injection, indicating that the BA/AH-DPI of the present application can obtain better therapeutic effect by BA/AH drug combination in combination with the pulmonary administration of DPI.

### In Vivo Pharmacokinetic Studies

### Laboratory Animals

SPF-grade healthy SD male rats, body weight 180-220 g, purchased from SPF (Beijing) Biotechnology Co., Ltd (Certificate No. SCXK (Beijing) 2019-0010), were conventionally reared under (23±2)° C., humidity: (50±10)%. This study was conducted with the approval of the Animal Experiment Center of Tianjin University of Traditional Chinese Medicine.

### Collection of Plasma Sample and Lung Tissue

108 SPF-grade SD male rats were randomly divided into tail intravenous injection group and pulmonary administration group, with 9 time points of 2 min, 4 min, 10 min, 15 min, 30 min, 1 h, 2 h, 4 h and 6 h per group, 6 rats per time point per group. The rats were fasted for 12 h before the experiment, with water provided. The rats were weighed (to calculate the dose) and numbered, and both groups were intraperitoneally injected with 10% chloral hydrate at a dose of 2 mL/kg. Group 1 was administered BA/AH-DPI saline solution by tail intravenous injection at a dose of 9 mg/kg. Group 2 was administered 3 mg of BA/AH-DPI fine powder at the same dose by spray administration with a DP-4 tracheal insufflator (1.5 mL of air was pushed each time, 3-4 times consecutively). Blood was sampled from the abdominal aorta of the rats in both groups at the above time points after drug administration, and the rats were executed. The obtained whole blood was placed in a centrifuge tube with sodium heparin, centrifuged at 4000 r/min for 10 min, and the upper layer of plasma was separated and stored a refrigerator at −80° C. After blood collection, the thoracic cavity was quickly opened and the lungs of the rats were isolated. The small amount of floating blood on the surface of the lung tissue was washed with saline in an ice bath. The surface of the lung tissue was blotted with filter paper. The trachea was cut off, wrapped in tin foil and placed in a 10 mL centrifuge tube. The lung was weighed precisely, and stored in a refrigerator at −80° C.

### Processing of Plasma Samples and Lung Tissue

The lung tissues were added to saline in a weight to volume ratio of 1:4 (g:mL). A homogenate was obtained by beating, dispersing and evenly grinding with a homogenizer under an ice water bath, and centrifuged at 6500 r/min for 20 min to obtain the supernatant of the lung tissue homogenate.

100 μL of the plasma sample or lung tissue supernatant was precisely absorbed, and 200 μL (100 ng/mL) of methanol solution containing the internal standard was added. The mixture was vortexed and mixed for 3 min, and centrifuged at 12000 r/min for 10 min to obtain the supernatant as the finished sample. The contents of BA and AH were determined by UPLC-MS/MS method.

### LC-MS Conditions

Chromatographic column: Waters ACQUITY UPLC® BEH C18 (100 mm×2.1 mm, 1.7 μm); mobile phase: 0.1% aqueous formic acid (A)-acetonitrile (B), gradient elution as shown in Table 11, column temperature: 30° C., flow rate: 0.3 mL/min, detection wavelength: 248 nm, injection volume: 5 μL, injection chamber temperature: 4° C.

TABLE 11
Gradient elution

| Time (min) | B (%) |
|---|---|
| 0 | 25 |
| 4 | 32 |
| 4.1 | 20 |
| 9 | 50 |
| 9.5 | 25 |
| 11 | 25 |

Electrospray ion source (ESI source); scan mode: positive (ESI+) detection; detection mode: multi-reactive ion monitoring (MRM). Electrospray voltage: 4 kV; atomization gas: N₂; spray gas flow rate (Gas1): 15 psi; ion source temperature: 300° C. The specific mass spectrometry parameters were shown in Table 12 below.

TABLE 12
Mass spectrometry parameter settings for the determination of
BA and AH in rat plasma and lung tissue homogenate

| | | Ion | Cleavage | | Collision |
|---|---|---|---|---|---|
| Compound | Mode | source | voltage (V) | MS1 → MS2 | energy (eV) |
| BA | MRM | EST⁺ | 100 | 447.09 → 270.8 | 38 |
| AH | MRM | EST⁺ | 145 | 378.9 → 263.6 | 34 |
| CBZ (1STD) | MRM | EST⁺ | 140 | 237.7 → 193.8 | 18 |

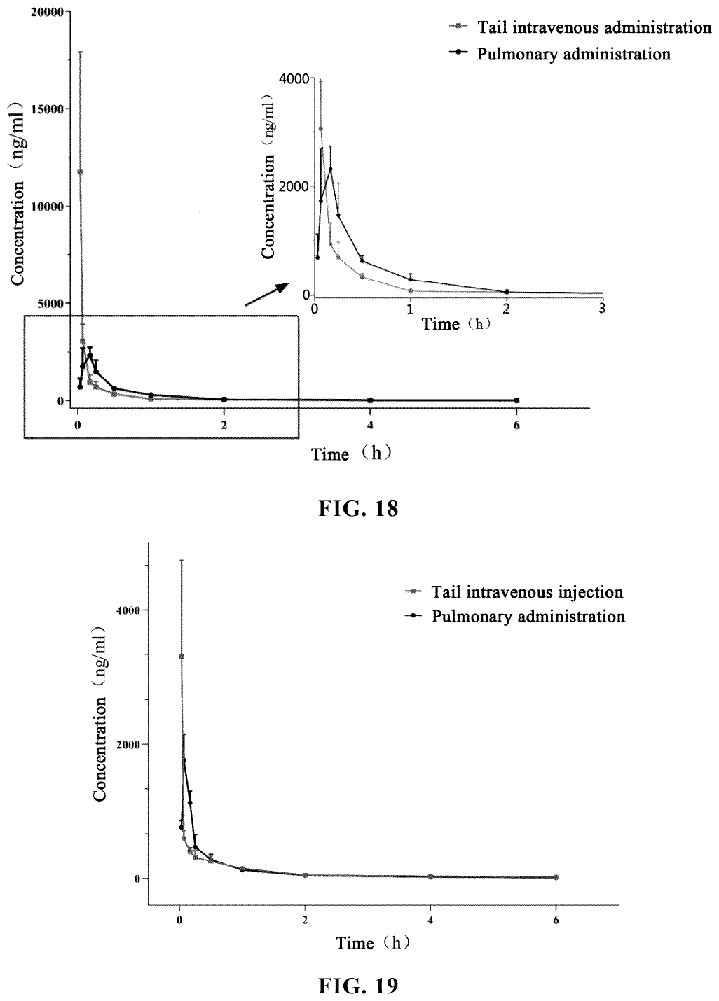

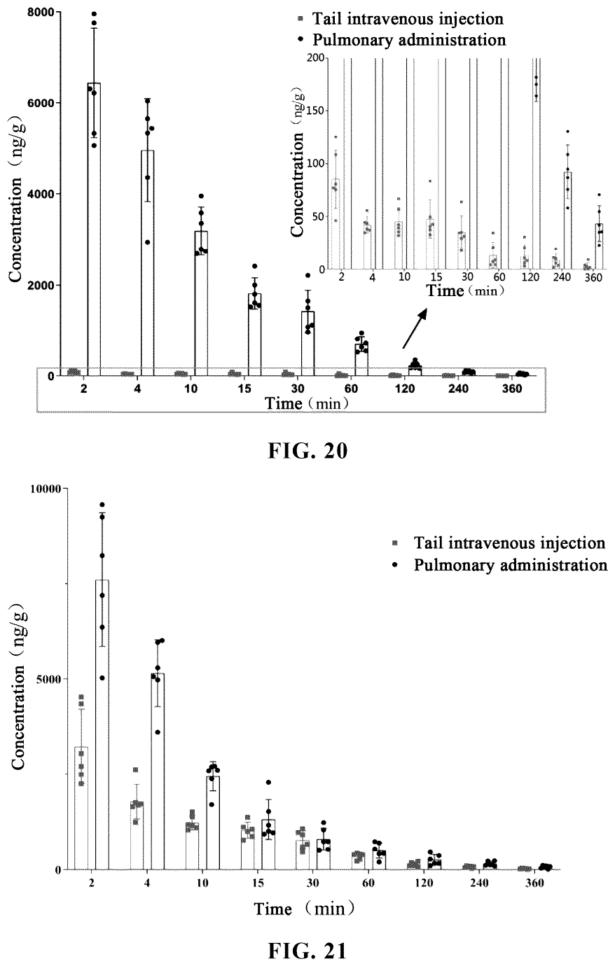

After pulmonary administration and tail intravenous injection administration of BA/AH-DPI of the present application, the results of the drug concentrations in rat plasma and lung tissue homogenate were shown in Table 13 and Table 14; the results of pharmacokinetic curves were shown in FIG. 18-FIG. 21, in which FIG. 18 showed the concentration curve of BA in plasma; FIG. 19 showed the concentration curve of AH in plasma; FIG. 20 showed the concentration curve of BA in lung tissue homogenate; FIG. 21 showed the concentration curve of AH in lung tissue homogenate; and the results of relevant pharmacokinetic parameters were shown in Table 15 and Table 16.

TABLE 13
Blood concentrations in the two groups of rats with pulmonary
administration and tail intravenous injection (ng/ml, Mean ± SD, n = 6)

| | Blood concentration (ng/ml) | | | |
|---|---|---|---|---|
| | Pulmonary | Pulmonary | Tail intravenous | |
| Time (min) | administration BA | administration AH | injection BA | Tail intravenous AH |
| 2 | 690.28 ± 437.59 | 762.85 ± 100.86 | 11741.55 ± 6174.18 | 3300.75 ± 1688.93 |
| 4 | 1738.10 ± 962.35 | 1765.74 ± 382.10 | 3064.27 ± 861.37 | 597.74 ± 232.64 |
| 10 | 2319.26 ± 421.35 | 1126.42 ± 170.08 | 933.33 ± 400.87 | 395.40 ± 141.57 |
| 15 | 1474.71 ± 588.42 | 464.22 ± 190.80 | 692.65 ± 283.74 | 306.27 ± 139.57 |
| 30 | 627.91 ± 95.86 | 279.57 ± 75.80 | 337.62 ± 70.28 | 251.75 ± 116.21 |
| 60 | 289.30 ± 109.52 | 123.83 ± 35.32 | 77 ± 46.12 | 148.08 ± 58.70 |
| 120 | 57.19 ± 14.24 | 40.17 ± 21.06 | 53.7 ± 54.23 | 49.44 ± 23.02 |
| 240 | 27.35 ± 10.90 | 21.04 ± 6.64 | 8.68 ± 8.46 | 33.85 ± 12.23 |
| 360 | 13.37 ± 3.68 | 8.57 ± 0.38 | 2.64 ± 5.27 | 18.92 ± 6.61 |

TABLE 14
Drug concentrations in lung tissue homogenates of the two
groups of rats with pulmonary administration and tail intravenous
injection (ng/ml, Mean ± SD, n = 6)

| | Drug concentration in lung tissue (ng/ml) | | | |
|---|---|---|---|---|
| | Pulmonary administration | Pulmonary Administration | Tail intravenous | Tail intravenous |
| Time (min) | BA | AH | injection BA | injection AH |
| 2 | 6439.59 ± 1202.15 | 7605.75 ± 1751.41 | 85.29 ± 27.93 | 3229.59 ± 973.41 |
| 4 | 4961.75 ± 1133.68 | 5152.11 ± 879.10 | 41.99 ± 7.67 | 1784.25 ± 453.98 |
| 10 | 3186.36 ± 524.04 | 2450.21 ± 384.07 | 45.09 ± 13.55 | 1228.94 ± 183.18 |
| 15 | 1815.23 ± 346.33 | 1315.45 ± 524.33 | 45.81 ± 18.70 | 1034.48 ± 209.99 |
| 30 | 1421.50 ± 469.26 | 798.39 ± 288.91 | 39.20 ± 22.04 | 779.99 ± 243.02 |
| 60 | 705.39 ± 159.55 | 502.68 ± 199.84 | 16.29 ± 19.54 | 352.09 ± 83.08 |
| 120 | 234.91 ± 76.15 | 255.50 ± 139.19 | 12.89 ± 10.47 | 134.87 ± 58.93 |
| 240 | 92.22 ± 25.45 | 152.98 ± 60.03 | 8.37 ± 6.37 | 68.42 ± 24.46 |
| 360 | 43.15 ± 16.91 | 66.19 ± 38.17 | 2.71 ± 3.37 | 23.38 ± 10.54 |

TABLE 15
Pharmacokinetic parameters of BA and AH in plasma after
pulmonary and tail intravenous injection in rats (ng/mL, Mean ± SD, n = 6)

| | | BA in plasma | | AH in plasma | |
|---|---|---|---|---|---|
| Pharmacokinetic | | Tail intravenous | Pulmonary | Tail intravenous | Pulmonary |
| parameters | Unit | injection group | administration group | injection group | administration group |
| HLz | h | 0.81 ± 0.60 | 2.01 ± 0.64** | 2.44 ± 0.89 | 1.82 ± 0.66 |
| Tₘₐₓ | h | 0.038 ± 0.01 | 0.15 ± 0.07 | 0.033 ± 0 | 0.067 ± 0 |
| Cₘₐₓ | ng/ml | 11812.90 ± 6050.73 | 2564.75 ± 351.04** | 3300.75 ± 1444.61 | 1765.74 ± 382.10* |
| AUC [symbol] | h * ng/ml | 1983.50 ± 1128.23 | 1203.63 ± 76.99 | 931.01 ± 422.07 | 632.84 ± 124.28 |
| AUC [symbol] | h * ng/ml | 2000.48 ± 1125.65 | 1244.70 ± 84.70 | 1001.09 ± 453.83 | 655.26 ± 123.20 |
| Vz/F | mL/kg | 7537.75 ± 8090.84 | 20858.49 ± 6574.83* | 34775.73 ± 16913.68 | 37204.14 ± 15553.42 |
| CL/F | mL/h/kg | 5908.83 ± 3723.21 | 7258.9 ± 499.08 | 10162.83 ± 3196.32 | 14195.61 ± 2992.02* |
| AUMC [symbol] | h * h * ng/ml | 396.82 ± 206.20 | 910.73 ± 152.90** | 716.00 ± 174.84 | 530.87 ± 152.15 |
| AUMC [symbol] | h * h * ng/ml | 496.81 ± 230.80 | 1289.05 ± 242.30** | 1421.37 ± 619.37 | 730.63 ± 145.82 |
| MRTlast | h | 0.24 ± 0.16 | 0.75 ± 0.092** | 0.82 ± 0.17 | 0.83 ± 0.10 |

Note:
Compared with tail intravenous injection group, *p < 0.05; **p < 0.01 [symbol]
indicates data missing or illegible when filed

TABLE 16
Pharmacokinetic parameters of BA and AH in lung tissue homogenates after pulmonary administration and tail intravenous
injection in rats (ng/mL, Mean ± SD, n = 6)

| | | BA in lung tissue | | AH in lung tissue | |
|---|---|---|---|---|---|
| Pharmacokinetic | | Tail intravenous | Pulmonary | Tail intravenous | Pulmonary |
| parameters | Unit | injection group | administration group | injection group | administration group |
| HLz | h | 1.69 ± 0.74 | 1.40 ± 0.40 | 1.21 ± 0.19 | 1.53 ± 0.44 |
| Tₘₐₓ | h | 0.033 ± 0 | 0.044 ± 0.018 | 0.033 ± 0 | 0.039 ± 0.013 |
| Cₘₐₓ | ng/g | 341.15 ± 111.71 | 26014.18 ± 4487.01** | 12918.35 ± 3893.62 | 30444.9 ± 6967.06** |
| AUC [symbol] | h * ng/g | 335.40 ± 164,99 | 11136.06 ± 1703.71** | 6159.13 ± 925.42 | 9900.01 ± 1801.11** |
| AUC [symbol] | h * ng/g | 344.81 ± 175.48 | 11136.06 ± 1703.71** | 6159.13 ± 925.42 | 9900.01 ± 1801.11** |
| Vz/F | g/kg | 78080.06 ± 69803.73 | 1630.13 ± 584.28* | 2489.18 ± 316.18 | 1883.19 ± 523.72* |
| CL/F | g/h/kg | 29086.90 ± 13876.33 | 800.14 ± 131.95** | 1454.72 ± 257.24 | 876.24 ± 146.55** |
| AUMC [symbol] | h * h * ng/g | 539.82 ± 360.54 | 10602.57 ± 1927.5 | 6303.88 ± 1584.34 | 12151.62 ± 4017.68** |
| AUMC [symbol] | h * h * ng/g | 870.60 ± 496.78 | 13424.36 ± 2111.98** | 7654.97 ± 2316.77 | 17898.55 ± 8080.42* |
| MRTlast | h | 1.52 ± 0.60 | 0.95 ± 0.087* | 1.01 ± 0.12 | 1.21 ± 0.20 |

Note:
Compared with tail intravenous injection group: *p < 0.05: **p < 0.01. [symbol]
indicates data missing or illegible when filed

Meaning of pharmacokinetic parameters: HLz was the biological half-life, i.e., the time required for the amount or blood concentration of the drug in the body to decrease by half; Tₘₐₓ was the time to peak, i.e., the time required to reach the peak concentration of the drug after administration. Cₘₐₓ was the peak drug concentration, i.e., the highest blood concentration that occurred after administration of the drug. AUC was the area enclosed by the blood drug concentration curve against the time axis. Vz/F was the apparent volume of distribution, the ratio constant of the amount of drug in the body to the blood concentration when the drug reached dynamic equilibrium in the body. CL/F was the clearance, i.e., the number of apparent volume of distribution of the drug removed from the body per unit time. AUMC was the first order moment of the drug-time curve, i.e. the area under the curse of the product of time and blood concentration versus time. MRTlast was the mean retention time, i.e., the average of the residence time of drug molecules in the body.

It followed from the results that compared with tail intravenous injection administration, after pulmonary administration of the same dose of BA/AH-DPI dry powder, AH reached maximum blood concentration in plasma at 4 min (Table 13) with a slightly larger drug retention time in vivo without a statistically significant differences (Table 15) and a relatively small AUC0-6 h for AH in the pulmonary administration group without statistically significant differences (Table 15), and the absolute bioavailability is 53.5% with high bioavailability (AUC). BA reached maximum blood concentration (Cₘₐₓ) in plasma around 9 min after pulmonary administration (p<0.01) (Table 15), with significantly longer in vivo half-life (p<0.01) and significantly longer mean retention time (MRTlast) in vivo (p<0.01) compared to tail intravenous injection (Table 15), all of which were significantly different. The half-life (HLz) of pulmonary administration BA was larger, the AUC 0-6 h was small but not statistically different (Table 15), and the absolute bioavailability reached 60.7% with high bioavailability. This showed that pulmonary administration could significantly increase the bioavailability of the drug in plasma and mean retention time (MRTlast) in vivo, and prolonged the half-life.

In lung tissue homogenates, the AUC0-6 h of BA in lung tissue was statistically highly significant (p<0.01) in the pulmonary administration group compared to the tail intravenous injection administration group, indicating that pulmonary administration significantly increased the bioavailability (AUC) of BA in lung tissue. BA showed a lower clearance in lung tissue and a relatively longer retention time in vivo (MRTlast) (Table 16), both with significant differences (p<0.05). Pulmonary administration AH showed relatively longer retention time of AH in lung tissue (MRTlast) (Table 16) compared to the tail intravenous injection group, without significant differences (p>0.05). However, the AUC0-6 h was significantly higher (p<0.01) (Table 16), the half-life (HLz) was prolonged (p>0.05), and the in vivo clearance (CL/F) was reduced (p<0.01) (Table 16), which were significantly different. It could be seen that pulmonary administration could significantly improve the intrapulmonary bioavailability of BA and AH with certain pulmonary targeting effect, which brought promising prospects for the treatment of pulmonary diseases.

The above examples are only the preferred examples of the present application and are not intended to limit the present application. Any modifications, equivalent substitutions and improvements made within the spirit and principle of the present application should be included in the protection scope of the present application.

1. A compound dry powder inhalant for treating idiopathic pulmonary fibrosis, consisting of baicalin, ambroxol hydrochloride, L-leucine and phosphate, wherein based on a mass of the compound dry powder inhalant, L-leucine accounts for 10-40%, phosphate accounts for 15-35%, and a total mass of baicalin and ambroxol hydrochloride accounts for 40-60%, and wherein a mass ratio of baicalin to ambroxol hydrochloride is 1:0.2 to 2; and the compound dry powder inhalant has a Dv90≤5 μm.

2. (canceled)

3. The compound dry powder inhalant according to claim 1, wherein based on the mass of the compound dry powder inhalant, L-leucine accounts for 15-25%.

4. The compound dry powder inhalant according to claim 1, wherein the compound dry powder inhalant has a Dv90≤3 μm.

5. The compound dry powder inhalant according to claim 1, wherein a fine particle fraction of baicalin is 30-60%; and a fine particle fraction of ambroxol hydrochloride is 25-50%.

6. The compound dry powder inhalant according to claim 1, wherein a mass median aerodynamic diameter of baicalin is 2-3.5 μm; and a mass median aerodynamic diameter of ambroxol hydrochloride is 2-3.5 μm.

7. The compound dry powder inhalant according to claim 1, wherein based on the mass of the compound dry powder inhalant, a moisture content is less than 7%.

8. The compound dry powder inhalant according to claim 1, wherein the compound dry powder inhalant is prepared by a spray drying.

9. The compound dry powder inhalant according to claim 8, wherein the spray drying comprises an inlet temperature of 75-85° C., an air flow of 80-120 L/min, a pump speed of 20-25%, a spray rate of 50-70%, and an internal pressure of 30-35 mbar.

10. (canceled)

11. A method of treating idiopathic pulmonary fibrosis, comprising administering an effective amount of the compound dry powder inhalant according to claim 1 to a subject in need thereof.

12. The compound dry powder inhalant according to claim 1, wherein the mass ratio of baicalin to ambroxol hydrochloride is 1:0.8 to 1.2.
